(19) United States
(12) Patent Application Publication
(10) Pub. No.: US 2024/0208652 A1
(43) Pub. Date: Jun. 27, 2024

(54) AIR MANAGEMENT SYSTEM WITH NEGATIVE ION TECHNOLOGY FOR IMPROVED AIR QUALITY

(71) Applicant: Hamilton Sundstrand Corporation, Charlotte, NC (US)

(72) Inventors: Venkatesh Komaragiri, Bangalore (IN); Sangati Surendra Reddy, Guntur (IN); Indramani Mohanty, Talcher (IN)

(21) Appl. No.: 18/184,841

(22) Filed: Mar. 16, 2023

(30) Foreign Application Priority Data
Dec. 21, 2022 (IN) 202211074226

(51) Int. Cl. B64D 13/06; A61L 9/22; B03C 3/011; B60H 3/00

(52) U.S. Cl. CPC B64D 13/06; A61L 9/22; B03C 3/011; B60H 3/0078; A61L 2209/14; A61L 2209/16; B03C 2201/30; B64D 2013/0618; B64D 2013/0644; B64D 2013/0648; B64D 2013/0688

(57) ABSTRACT

The present disclosure provides for air management systems, assemblies and methods. More particularly, the present disclosure provides for air management systems, assemblies and methods (e.g., for aircraft or the like), with the air management systems/assemblies having negative ion technology for improved air quality. The present disclosure provides improved air conditioning/management systems having features to handle the poisonous/toxic chemicals/gases (e.g., carbon monoxide), climate relevant gases (e.g., methane), and/or harmful bacteria/viruses (e.g., SARS-CoV-2), and to improve the air quality inside the aircraft through the disclosed air conditioning/management systems.

### CROSS-REFRENCE TO RELATED APPLICATIONS

This application claims the benefit of IN Application No. 202211074226 filed Dec. 21, 2022, the disclosure of which is incorporated herein by reference in its entirety.

### TECHNICAL FIELD

The present disclosure relates to air management systems, assemblies and methods and, more particularly, to air management systems, assemblies and methods (e.g., for aircraft or the like), with the air management systems/assemblies having negative ion technology for improved air quality.

### BACKGROUND

In general, the cabin air supply on most large commercial aircraft is provided using engine bleed air systems. There is evidence that some people experience acute symptoms due to a fume event or exposure to contaminants in aircraft cabin air.

Currently available air management systems typically cannot handle the toxic chemicals/gases and/or harmful viruses/bacteria, and a significant percentage of the viruses will pass through the filters (e.g., high-efficiency particulate air or HEPA filters). As such, this may cause the outbreak of harmful viruses/bacteria (e.g., SARS-CoV-2 or the like).

### BRIEF DESCRIPTION

The present disclosure provides for air management systems, assemblies and methods. More particularly, the present disclosure provides for air management systems, assemblies and methods (e.g., for aircraft or the like), with the air management systems/assemblies having negative ion technology for improved air quality.

The present disclosure provides for an air management system including a mixer unit in fluid communication with an aircraft cabin, and the mixer unit in fluid communication with a negative ion generator and with filtered air from the aircraft cabin; wherein the negative ion generator is configured to generate negative ions that mix with the filtered air from the aircraft cabin in the mixer unit, and the mixer unit is configured to send the mixed air to the aircraft cabin; and wherein after joint union or absorption of the generated negative ions with positive ions from the mixed air, the joint union or absorption of the generated negative ions with the positive ions inhibits growth of bacterial or virus activity and reduces concentrations of toxic gases or chemicals in the mixed air in the aircraft cabin.

In addition to one or more of the features described, or as an alternative to any of the foregoing embodiments, the negative ion generator is configured to generate negative hydroxyl radicals or ions.

In addition to one or more of the features described, or as an alternative to any of the foregoing embodiments, the joint union or absorption of the generated negative ions with the positive ions inhibits growth of SARS-CoV-2 in the mixed air in the aircraft cabin.

In addition to one or more of the features described, or as an alternative to any of the foregoing embodiments, the joint union or absorption of the generated negative ions with the positive ions reduces concentrations of at least one of smoke, vapor, haze, fumes, dust, allergens, abnormal odors, carbon monoxide, methane or tricresyl phosphate in the mixed air in the aircraft cabin.

In addition to one or more of the features described, or as an alternative to any of the foregoing embodiments, the joint union or absorption of the generated negative ions with the positive ions inhibits growth of microbes or airborne bacteria.

In addition to one or more of the features described, or as an alternative to any of the foregoing embodiments, the negative ion generator is configured to generate nano-sized capsules of water enveloping amounts of negative hydroxyl radicals or ions.

In addition to one or more of the features described, or as an alternative to any of the foregoing embodiments, the negative hydroxyl radicals or ions react by transforming hydrogen from components in the mixed air into water.

In addition to one or more of the features described, or as an alternative to any of the foregoing embodiments, the mixer unit is in fluid communication with a cooled air stream from a turbine of an air cycle machine.

In addition to one or more of the features described, or as an alternative to any of the foregoing embodiments, the air cycle machine comprises a compressor, and the compressor is in fluid communication with an air feed stream; and the air feed stream comprises a bleed air feed stream from a bleed air system or comprises an ambient air feed stream from a no-bleed system.

In addition to one or more of the features described, or as an alternative to any of the foregoing embodiments, the turbine is in fluid communication with a ram air stream that is fed to the turbine via at least one main heat exchanger.

The present disclosure provides for a method for operating an air management system including providing a mixer unit in fluid communication with an aircraft cabin, and the mixer unit in fluid communication with a negative ion generator and with filtered air from the aircraft cabin; and generating negative ions via the negative ion generator; and mixing the generated negative ions with the filtered air from the aircraft cabin in the mixer unit; and sending the mixed air to the aircraft cabin via the mixer unit; and wherein after joint union or absorption of the generated negative ions with positive ions from the mixed air, the joint union or absorption of the generated negative ions with the positive ions inhibits growth of bacterial or virus activity and reduces concentrations of toxic gases or chemicals in the mixed air in the aircraft cabin.

In addition to one or more of the features described, or as an alternative to any of the foregoing embodiments, the negative ion generator generates negative hydroxyl radicals or ions.

In addition to one or more of the features described, or as an alternative to any of the foregoing embodiments, the joint union or absorption of the generated negative ions with the positive ions inhibits growth of SARS-CoV-2 in the mixed air in the aircraft cabin.

In addition to one or more of the features described, or as an alternative to any of the foregoing embodiments, the joint union or absorption of the generated negative ions with the positive ions reduces concentrations of at least one of smoke, vapor, haze, fumes, dust, allergens, abnormal odors, carbon monoxide, methane or tricresyl phosphate in the mixed air in the aircraft cabin.

In addition to one or more of the features described, or as an alternative to any of the foregoing embodiments, the joint union or absorption of the generated negative ions with the positive ions inhibits growth of microbes or airborne bacteria.

In addition to one or more of the features described, or as an alternative to any of the foregoing embodiments, the negative ion generator generates nano-sized capsules of water enveloping amounts of negative hydroxyl radicals or ions.

In addition to one or more of the features described, or as an alternative to any of the foregoing embodiments, the negative hydroxyl radicals or ions react by transforming hydrogen from components in the mixed air into water.

In addition to one or more of the features described, or as an alternative to any of the foregoing embodiments, the mixer unit is in fluid communication with a cooled air stream from a turbine of an air cycle machine.

In addition to one or more of the features described, or as an alternative to any of the foregoing embodiments, the air cycle machine comprises a compressor, and the compressor is in fluid communication with an air feed stream; and the air feed stream comprises a bleed air feed stream from a bleed air system or comprises an ambient air feed stream from a no-bleed system.

In addition to one or more of the features described, or as an alternative to any of the foregoing embodiments, the turbine is in fluid communication with a ram air stream that is fed to the turbine via at least one main heat exchanger.

The above described and other features are exemplified by the following figures and detailed description.

Any combination or permutation of embodiments is envisioned. Additional features, functions and applications of the disclosed systems, assemblies and methods of the present disclosure will be apparent from the description which follows, particularly when read in conjunction with the appended figures. All references listed in this disclosure are hereby incorporated by reference in their entireties.

### BRIEF DESCRIPTION OF THE DRAWINGS

The following figures are example embodiments wherein the like elements are numbered alike.

Features and aspects of embodiments are described below with reference to the accompanying drawings, in which elements are not necessarily depicted to scale.

Example embodiments of the present disclosure are further described with reference to the appended figures. It is to be noted that the various features, steps, and combinations of features/steps described below and illustrated in the figures can be arranged and organized differently to result in embodiments which are still within the scope of the present disclosure. To assist those of ordinary skill in the art in making and using the disclosed systems, assemblies and methods, reference is made to the appended figures, wherein:

FIG. 1 is a schematic of an example air management system, according to the present disclosure; and

FIG. 2 is a partial schematic from an example air management system, according to the present disclosure.

### DETAILED DESCRIPTION

The example embodiments disclosed herein are illustrative of air management systems, and assemblies of the present disclosure and methods/techniques thereof. It should be understood, however, that the disclosed embodiments are merely examples of the present disclosure, which may be embodied in various forms. Therefore, details disclosed herein with reference to example air management systems and associated processes/techniques of fabrication/assembly and use are not to be interpreted as limiting, but merely as the basis for teaching one skilled in the art how to make and use the systems/assemblies and/or alternative systems/assemblies of the present disclosure.

The present disclosure provides for air management systems, assemblies and methods. More particularly, the present disclosure provides for air management systems, assemblies and methods (e.g., for aircraft or the like), with the air management systems/assemblies having negative ion technology for improved air quality.

In general, current practice provides that some available air conditioning/management systems in aircraft cannot handle fume events (e.g., abnormal odors, smoke, haze or fumes in the cabin) that may arise from various internal or external sources, and some may be due to contamination of the bleed air supply (e.g., as a result of a failure of an oil seal in the engine). Moreover, some available air conditioning/management systems in aircraft cannot handle harmful viruses/bacteria efficiently.

The present disclosure provides improved air conditioning/management systems having features to handle the poisonous/toxic chemicals/gases (e.g., carbon monoxide), climate relevant gases (e.g., methane), and/or harmful bacteria/viruses (e.g., SARS-CoV-2), and to improve the air quality inside the aircraft through the disclosed air conditioning/management systems.

As noted, some people experience acute symptoms due to a fume event or exposure to contaminants in conventional aircraft cabin air. Some of the chemical contaminants that are present during such events are irritants, and may cause itching or soreness of the eyes, nasal discharge, sore throat or coughing.

For example, turbine engine oil is an irritant and can include neurotoxic chemicals (e.g., tricresyl phosphate). Moreover, hydraulic fluid, although typically non-toxic in small quantities, can be extremely irritating to the eyes and skin, and can create a hazard to pilots during a fume event (but may cause no lasting damage).

Furthermore, deicing fluid has a strong smell, but is not very irritating or toxic if inhaled (as opposed to the significant toxicity when ingested).

A U.S. Federal Aviation Administration (FAA) funded study found that fume events occur on 1 in 5,000 flights, and that some planes may have multiple consecutive events if the leak is not fixed. Airlines are required to file Service Difficulty Reports (SDRs) when smoke, vapor or noxious odors enter the cockpit or passenger cabin. In 2018, U.S. airlines conducted more than 12 million flights. The FAA's SDR database showed 232 submissions from air carriers that referenced a fume event that year.

The FAA requires manufacturers to show that the airplane crew and passenger compartment air is free from harmful or hazardous concentrations of gases and vapors.

As noted, currently available air conditioning/management systems in aircraft cannot handle the poisonous/toxic chemicals/gases and/or harmful viruses/bacteria efficiently. Currently used HEPA filters in cabin air conditioning and temperature control systems cannot trap/kill all of the viruses, and a significant percentage of the viruses will pass through these HEPA filters and circulate back into the cabin. In some cases, some of the trapped viruses on the HEPA filter will be blown through the HEPA filter into the cabin with the next wave of air. Due to the long-surviving time of viruses like SARS-CoV-2 in air, these particles linger for anywhere up to several hours, and this may cause the outbreak of harmful viruses/bacteria.

The present disclosure provides for air management systems, assemblies and methods (e.g., for aircraft or the like), with the air management systems/assemblies having negative ion technology for improved air quality.

In example embodiments and as discussed further below, the air management systems of the present disclosure have a negative ion generator that generates negative ions that mix with the cabin air. As such and after joint union/absorption of generated negative ions with positive ions (e.g., dust, microbes, airborne bacteria, allergens, viruses, etc.), the joint union/absorption of generated negative ions with positive ions become too heavy to float in the air and they will fall to the ground. With respect to smoke, odors, and/or poisonous/toxic chemicals, the joint union of these components with the generated negative ions will cause these components to become chemically diluted.

In example embodiments, the air management systems of the present disclosure have a negative ion generator that generates negative hydroxyl radicals or ions (OH- ions).

For example, the negative ion generator can collect (invisible) moisture in the air (or water), and can apply high voltage to it, thereby generating capsules of water including an abundance of (e.g., huge volumes of) negative OH- radicals/ions. Nano-sized capsules of water enveloping large amounts of negative OH- radicals/ions can be released all together into the air. The more negative OH- radicals/ions there are, then more bacteria/viruses and/or poisonous/toxic chemicals/gases in the cabin air can be effectively inhibited.

As the released nano-sized capsules of water (e.g., including large volumes of negative OH- radicals/ions) reach the pollutants in the air, the negative OH- radicals/ions react by transforming hydrogen from the pollutants into water, and this helps to inhibit growth of bacterial activity and/or reduces the concentrations of toxic gases/chemicals in the air.

It is noted that negative OH- radicals/ions have outstanding performance on odor removal and can inhibit activity of bacteria and viruses, and the negative OH- radicals/ions can control the concentrations of toxic gases (e.g., carbon monoxide and/or climate relevant gases such as methane or the like).

As discussed further below, the example air management/conditioning systems of the present disclosure can spread these nano-sized capsules of water (e.g., containing large volumes of negative OH- radicals/ions) by air flow of the internal air conditioners inside the aircraft or the like.

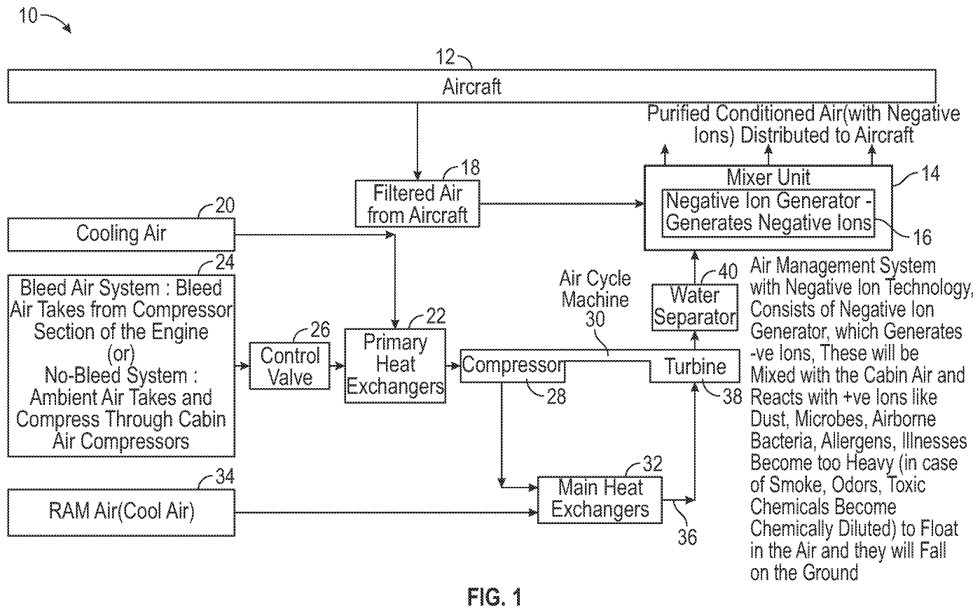

FIG. 1 is a schematic of an example air management system 10 of the present disclosure. In general, air management system 10 is an air management system 10 for aviation (e.g., for aircraft or the like), with the air management system 10 having negative ion technology for improved air quality, as discussed further below.

As shown in FIG. 1, the air management system 10 includes an aircraft cabin 12 in fluid communication with a mixer unit 14. The mixer unit 14 is in fluid communication with a negative ion generator 16, and the mixer unit 14 is in fluid communication with filtered air 18 from the aircraft cabin 12.

In example embodiments, the air management system 10 includes a stream of cooling air 20 in fluid communication with at least one primary heat exchanger 22, and an air feed stream 24 in fluid communication with the at least one primary heat exchanger 22 via a control valve 26.

It is noted that in some embodiments, the air feed stream 24 can be a bleed air feed stream 24 from a bleed air system, where bleed air is provided by a compressor section of the aircraft engine. In other embodiments, the air feed stream 24 can be an ambient air feed stream 24 provided by a no-bleed system where ambient air is compressed through one or more cabin air compressors.

As shown in FIG. 1, the air feed stream 24 and the cooling air stream 20 are fed to a compressor 28 of an air cycle machine 30. The compressor 28 then compresses the streams 20, 24, and feeds the compressed air to at least one main heat exchanger 32. A ram air stream 34 is also fed to the at least one main heat exchanger 32. After the at least one main heat exchanger 32, a cooled air stream 36 is fed to a turbine 38 of the air cycle machine 30, and then is fed to a water separator 40 and then to the mixer unit 14.

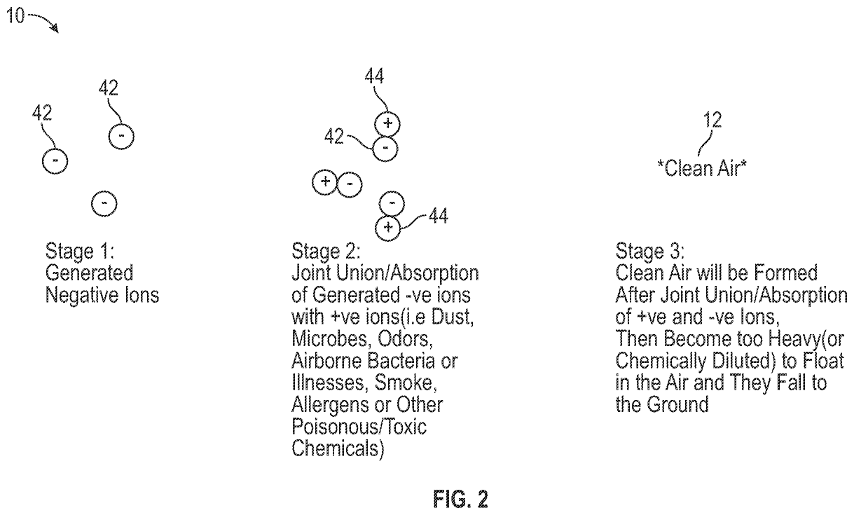

In general and as also shown in FIG. 2, the negative ion generator 16 generates negative ions 42 that mix with the filtered cabin air 18 in the mixer unit 14 and with the cooled air stream 36 fed to the mixer unit 14. FIG. 2 is a partial schematic from an example air management system 10, according to the present disclosure.

As such and after joint union/absorption of generated negative ions 42 with positive ions 44 (e.g., dust, microbes, airborne bacteria, allergens, viruses, etc.) from the filtered cabin air 18 and from air in the cabin 12, the joint union/absorption of generated negative ions 42 with positive ions 44 become too heavy to float in the air in the cabin 12 and they will fall to the ground. With respect to smoke, odors, and/or poisonous/toxic chemicals, the joint union of these components 44 with the generated negative ions 42 will cause these components to become chemically diluted in the air in the cabin 12.

In example embodiments, the negative ion generator 16 generates negative hydroxyl radicals or ions (OH- ions) 42. For example, the negative ion generator 16 can collect (invisible) moisture in the air 18, 36 (or water), and can apply high voltage to it, thereby generating capsules of water including an abundance of (e.g., large volumes of) negative OH- radicals/ions 42. Nano-sized capsules of water enveloping large amounts of negative OH- radicals/ions 42 can be released all together into the air via the mixer unit 14 and sent to the cabin 12. The more negative OH- radicals/ions 42 there are, then more bacteria/viruses and/or poisonous/toxic chemicals/gases in the air in the cabin 12 can be effectively inhibited.

As the released nano-sized capsules of water (e.g., including large volumes of negative OH- radicals/ions 42) reach the pollutants in the air, the negative OH- radicals/ions 42 react by transforming hydrogen from the pollutants into water, and this helps to inhibit growth of bacterial/virus activity and/or reduces the concentrations of toxic gases/chemicals in the air in the cabin 12. The negative OH- radicals/ions 42 have outstanding performance on odor removal and can inhibit activity of bacteria and viruses, and the negative OH- radicals/ions 42 can control the concentrations of toxic gases (e.g., carbon monoxide and/or climate relevant gases such as methane or the like) in the air of cabin 12.

As such and as shown in FIG. 1, the example air management system 10 can spread these nano-sized capsules of water (e.g., containing large volumes of negative OH- radicals/ions 42) by air flow of the internal air conditioners inside the aircraft of system 10.

There are many benefits of the systems, assemblies and methods of the present disclosure, including, without limitation, improving the air quality inside the aircraft through the air management systems by controlling the concentration of poisonous/toxic chemicals/gases (e.g., carbon monoxide and/or climate relevant gases like methane or the like); the air management systems facilitate with odor removal and/or inhibit activity of bacteria and/or viruses; the air management systems can avoid the outbreak of harmful viruses like SARS-CoV-2 (which has long-surviving time in air); and/or the air management systems can avoid or reduce the effects of fume events.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

The ranges disclosed herein are inclusive of the endpoints, and the endpoints are independently combinable with each other (e.g., ranges of “up to 25 wt. %, or, more specifically, 5 wt. % to 20 wt. %”, is inclusive of the endpoints and all intermediate values of the ranges of “5 wt. % to 25 wt. %,” etc.). “Combinations” is inclusive of blends, mixtures, alloys, reaction products, and the like. The terms “first,” “second,” and the like, do not denote any order, quantity, or importance, but rather are used to distinguish one element from another. The terms “a” and “an” and “the” do not denote a limitation of quantity and are to be construed to cover both the singular and the plural, unless otherwise indicated herein or clearly contradicted by context. “Or” means “and/or” unless clearly stated otherwise. Reference throughout the specification to “some embodiments”, “an embodiment”, and so forth, means that a particular element described in connection with the embodiment is included in at least one embodiment described herein, and may or may not be present in other embodiments. In addition, it is to be understood that the described elements may be combined in any suitable manner in the various embodiments. A “combination thereof” is open and includes any combination comprising at least one of the listed components or properties optionally together with a like or equivalent component or property not listed.

Unless defined otherwise, technical and scientific terms used herein have the same meaning as is commonly understood by one of skill in the art to which this application belongs. All cited patents, patent applications, and other references are incorporated herein by reference in their entirety. However, if a term in the present application contradicts or conflicts with a term in the incorporated reference, the term from the present application takes precedence over the conflicting term from the incorporated reference.

Although the systems and methods of the present disclosure have been described with reference to example embodiments thereof, the present disclosure is not limited to such example embodiments and/or implementations. Rather, the assemblies, systems and methods of the present disclosure are susceptible to many implementations and applications, as will be readily apparent to persons skilled in the art from the disclosure hereof. The present disclosure expressly encompasses such modifications, enhancements and/or variations of the disclosed embodiments. Since many changes could be made in the above construction and many widely different embodiments of this disclosure could be made without departing from the scope thereof, it is intended that all matter contained in the drawings and specification shall be interpreted as illustrative and not in a limiting sense. Additional modifications, changes, and substitutions are intended in the foregoing disclosure. Accordingly, it is appropriate that the appended claims be construed broadly and in a manner consistent with the scope of the disclosure.

What is claimed is:

1. An air management system comprising:
a mixer unit in fluid communication with an aircraft cabin, and the mixer unit in fluid communication with a negative ion generator and with filtered air from the aircraft cabin;
wherein the negative ion generator is configured to generate negative ions that mix with the filtered air from the aircraft cabin in the mixer unit, and the mixer unit is configured to send the mixed air to the aircraft cabin; and
wherein after joint union or absorption of the generated negative ions with positive ions from the mixed air, the joint union or absorption of the generated negative ions with the positive ions inhibits growth of bacterial or virus activity and reduces concentrations of toxic gases or chemicals in the mixed air in the aircraft cabin.

2. The system of claim 1, wherein the negative ion generator is configured to generate negative hydroxyl radicals or ions.

3. The system of claim 1, wherein the joint union or absorption of the generated negative ions with the positive ions inhibits growth of SARS-CoV-2 in the mixed air in the aircraft cabin.

4. The system of claim 1, wherein the joint union or absorption of the generated negative ions with the positive ions reduces concentrations of at least one of smoke, vapor, haze, fumes, dust, allergens, abnormal odors, carbon monoxide, methane or tricresyl phosphate in the mixed air in the aircraft cabin.

5. The system of claim 1, wherein the joint union or absorption of the generated negative ions with the positive ions inhibits growth of microbes or airborne bacteria.

6. The system of claim 1, wherein the negative ion generator is configured to generate nano-sized capsules of water enveloping amounts of negative hydroxyl radicals or ions.

7. The system of claim 6, wherein the negative hydroxyl radicals or ions react by transforming hydrogen from components in the mixed air into water.

8. The system of claim 1, wherein the mixer unit is in fluid communication with a cooled air stream from a turbine of an air cycle machine.

9. The system of claim 8, wherein the air cycle machine comprises a compressor, and the compressor is in fluid communication with an air feed stream; and
wherein the air feed stream comprises a bleed air feed stream from a bleed air system or comprises an ambient air feed stream from a no-bleed system.

10. The system of claim 8, wherein the turbine is in fluid communication with a ram air stream that is fed to the turbine via at least one main heat exchanger.

11. A method for operating an air management system comprising:
providing a mixer unit in fluid communication with an aircraft cabin, and the mixer unit in fluid communication with a negative ion generator and with filtered air from the aircraft cabin;
generating negative ions via the negative ion generator;
mixing the generated negative ions with the filtered air from the aircraft cabin in the mixer unit;
sending the mixed air to the aircraft cabin via the mixer unit; and
wherein after joint union or absorption of the generated negative ions with positive ions from the mixed air, the joint union or absorption of the generated negative ions with the positive ions inhibits growth of bacterial or virus activity and reduces concentrations of toxic gases or chemicals in the mixed air in the aircraft cabin.

12. The method of claim 11, wherein the negative ion generator generates negative hydroxyl radicals or ions.

13. The method of claim 11, wherein the joint union or absorption of the generated negative ions with the positive ions inhibits growth of SARS-CoV-2 in the mixed air in the aircraft cabin.

14. The method of claim 11, wherein the joint union or absorption of the generated negative ions with the positive ions reduces concentrations of at least one of smoke, vapor, haze, fumes, dust, allergens, abnormal odors, carbon monoxide, methane or tricresyl phosphate in the mixed air in the aircraft cabin.

15. The method of claim 11, wherein the joint union or absorption of the generated negative ions with the positive ions inhibits growth of microbes or airborne bacteria.

16. The method of claim 11, wherein the negative ion generator generates nano-sized capsules of water enveloping amounts of negative hydroxyl radicals or ions.

17. The method of claim 16, wherein the negative hydroxyl radicals or ions react by transforming hydrogen from components in the mixed air into water.

18. The method of claim 11, wherein the mixer unit is in fluid communication with a cooled air stream from a turbine of an air cycle machine.

19. The method of claim 18, wherein the air cycle machine comprises a compressor, and the compressor is in fluid communication with an air feed stream; and
wherein the air feed stream comprises a bleed air feed stream from a bleed air system or comprises an ambient air feed stream from a no-bleed system.

20. The method of claim 18, wherein the turbine is in fluid communication with a ram air stream that is fed to the turbine via at least one main heat exchanger.
